(19) United States
(12) Patent Application Publication
(10) Pub. No.: US 2017/0143192 A1
(43) Pub. Date: May 25, 2017

(54) Fabric Tube Propulsion Drive

(71) Applicant: Daniel Nagase, Vancouver (CA)

(72) Inventor: Daniel Nagase, Vancouver (CA)

(21) Appl. No.: 14/949,882

(22) Filed: Nov. 24, 2015

(51) Int. Cl. A61B 1/00; A61B 1/018; A61B 1/04

(52) U.S. Cl. CPC A61B 1/00133; A61B 1/04; A61B 1/018; A61B 1/00137

(57) ABSTRACT

Fabric Tube Propulsion Drive is a system for propelling medical devices through hollow body organs and within body cavities without causing trauma to the body surfaces upon which it operates. Fabric Tube Propulsion Drive consists of a hollow tube surrounded by a continuous loop of elastic fabric or mesh. Within the tube is a motor drive system that moves the fabric through the lumen of the tube such that the fabric on the outside of the tube can continuously interface with the body part through which the Fabric Tube Propulsion Drive is operating, and thereby drive the whole device through the body cavity or over the surface in question. Furthermore, the motor drive system can selectively apply tension to the fabric over specific areas within the tube to cause flexion of the whole tube.

### CROSS-REFERENCE TO RELATED APPLICATIONS

U.S. Pat. No. 8,694,092

Ferren, et al. Apr. 8, 2014

Lumen-traveling biological interface device and method of use

U.S. Pat. No. 8,771,175

Schostek, et al. Jul. 8, 2014

Capsule type endoscope including magnetic drive

U.S. Pat. No. 8,328,714

Couvillon, Jr. Dec. 11, 2012

Robotic endoscope

U.S. Pat. No. 8,245,799

Chiel, et al. Aug. 21, 2012

Apparatus and method for locomotion

U.S. Pat. No. 8,214,019

Govari, et al. Jul. 3, 2012

Robotically guided catheter

U.S. Pat. No. 7,857,767

Ferren, et al. Dec. 28, 2010

Lumen-traveling device

U.S. Pat. No. 7,708,687

Bern, et al. May 4, 2010

Endoscope propulsion system and method

U.S. Pat. No. 7,666,135

Couvillon, Jr. Feb. 23, 2010

Robotic endoscope

U.S. Pat. No. 6,988,986

Gross Jan. 24, 2006

Self-propelled imaging system

U.S. Pat. No. 5,337,732

Grundfest, et al. Aug. 16, 1994

Robotic endoscopy

U.S. Pat. No. 6,764,441

Chiel, et al. Jul. 20, 2004

Peristaltically self-propelled endoscopic device

U.S. Pat. No. 8,827,948

Romo, et al. Sep. 9, 2014

Steerable catheters

U.S. Pat. No. 8,882,660

Phee, et al. Nov. 11, 2014

Robotic system for flexible endoscopy

U.S. Pat. No. 8,870,912

Brisson, et al. Oct. 28, 2014

Surgical instrument with single drive input for two end effector mechanisms

U.S. Pat. No. 6,332,865

Borody, et al. Dec. 25, 2001

Self-advancing endoscope

U.S. Pat. No. 7,918,786

Kawano, et al. Apr. 5, 2011

Capsule type medical device system, and capsule type medical device

U.S. Pat. No. 8,449,454

Takizawa May 28, 2013

Capsule endoscope system

U.S. Pat. No. 8,496,573

Hata, et al. Jul. 30, 2013

Steerable capsule apparatus and method

### STATEMENT REGARDING FEDERALLY SPONSORED RESEARCH OR DEVELOPMENT (IF APPLICABLE)

Not Applicable

### REFERENCE TO SEQUENCE LISTING, A TABLE, OR A COMPUTER PROGRAM LISTING COMPACT DISC APPENDIX (IF APPLICABLE)

Not Applicable

### BACKGROUND OF THE INVENTION

Patent literature is well populated with various methods of propulsion for objects and tools within the human or animal body. Primarily, propulsion patents relate to laproscopy and endoscopy—either tethered as in a standard endoscope for exploration, manipulation and surgery, or untethered as in the case for robotic endoscopy and laproscopy. However, propulsion within a living organism presents numerous challenges. First, traction on cavitary surfaces can be low due to the presence of mucus, blood or other fluid. Second, orientation in space with respect to gravity is highly variable. Third is the high variability in terms of shape of the area being explored. The multitude of existing and patent proposed propulsion systems have various drawbacks such as complexity, slow movement, or mechanical harm to the organ or body cavity from propulsion system operation. The subject of this patent describes a novel yet simple fabric tube propulsion drive system capable of traversing any body cavity or hollow body structure with speed and minimal abrasive trauma. Furthermore this drive system is also capable of operating within pools of liquid when no surface contact is available. This drive system is primarily directed at but not limited to operation within the digestive tract, hollow body cavities, and airways of the of the lungs.

### RATIONALE

For a propulsion system to be useful and effective within a human or animal body, it must first cause little to no damage to the body within which it operates. It must also be capable of traversing both liquid and air filled structures, as well as elastic structures with variable volumes such as the intestine and stomach. Many digestive tract organs are elastic and can have very small or even no internal volume until an object or substance is introduced within it. e.g Food, endoscope, or a robotic device. An ideal propulsion system should be capable of opening such closed spaces with minimal trauma to the body tissue. Furthermore such a propulsion system should also be capable of moving quickly through such spaces without causing abrasion to the surfaces upon which it travels.

### BRIEF SUMMARY OF THE INVENTION

The Fabric Tube Propulsion Drive is partly inspired by the shape and functional characteristics of a sea cucumber. It consists of 3 functional parts.

1) A flexible tubelike endoskeleton that provides moderate longitudinal rigidity while allowing the device to flex and bend in planes that are perpendicular to it's longitudinal axis. This flexible endoskeleton may be thought of as the internal “frame” of the device.

2) A continuous loop of elastic fabric or mesh that surrounds both the inner and outer surfaces of the endoskeleton/frame. The fabric or mesh can have various textures to allow for effective traction and propulsion on specific body surfaces. The ideal such surface texture described within this patent is a mesh or fabric surface with “pile” similar to the tufts of “pile” that cover carpet flooring. Such a surface allows for propulsion on slippery surfaces and through liquids by providing a high surface area while on the outside of tubelike body of the device. As the fabric traverses into the inner surface of the tubelike body of the device, the “pile” is compacted producing less surface area and therefore less propulsion opposite to the propulsion forces made on the outside of the device.

3) An internal motor drive system that sits within the tube of the endoskeleton frame that is mechanically held within the frame by narrowings at either end of said endoskeleton frame. This motor drive system can bend the endoskeleton or frame of the device by varying the tension applied to various portions of the fabric covering the inner surface of the endoskeleton.

### BRIEF DESCRIPTION OF THE SEVERAL VIEWS OF THE DRAWING

FIG. 1: Overall external view of the Fabric Tube Propulsion Drive system.

FIG. 2: Cutaway view of one possible endoskeleton design.

FIG. 3: Cross section of the Fabric Tube Propulsion Drive with the position of the motor drive system shown within the endoskeleton

FIG. 4: A semi transparent 3d view of the device with the appoximate location of the motor drive wheels within the device.

FIG. 5: A semi transparent 3d view of the device with the overlying fabric surrounding both the inner and outer surfaces of the endoskeleton.

FIG. 6: A 2 dimensional cut-away view showing the rotation of the wheels and fabric tensions necessary to cause flexion of the endoskeleton. The curved arrows show the rotation of the wheels while the straight heavy arrows show the direction of movement of the endoskeleton. The straight regular arrows show the direction of fabric tension and compression.

### DETAILED DESCRIPTION OF THE INVENTION

The figures and following text describe the longitudinally shortest implementation of the Fabric Tube Propulsion Drive. However, variations of the design can easily accommodate any length and width of device necessary to contain video equipment, surgical tools and energy sources to power the system. Alternatively, the drive system may be operated on a tether to obviate the need for an internal power source during endoscopy or laproscopic surgery. A tethered implementation has certain advantages as it can also enable difficult to implement functions in a device that is independent of any connection outside the body, e.g. continuous suction.

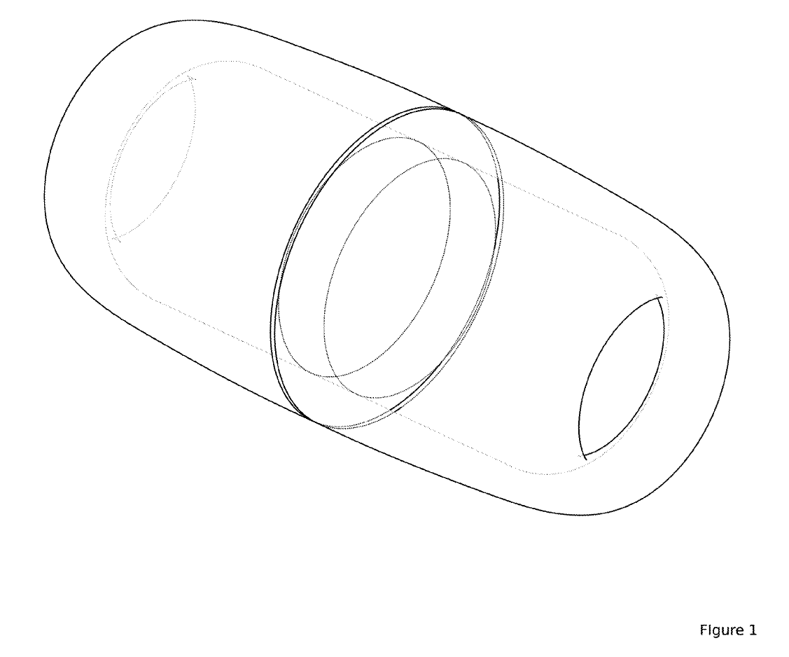

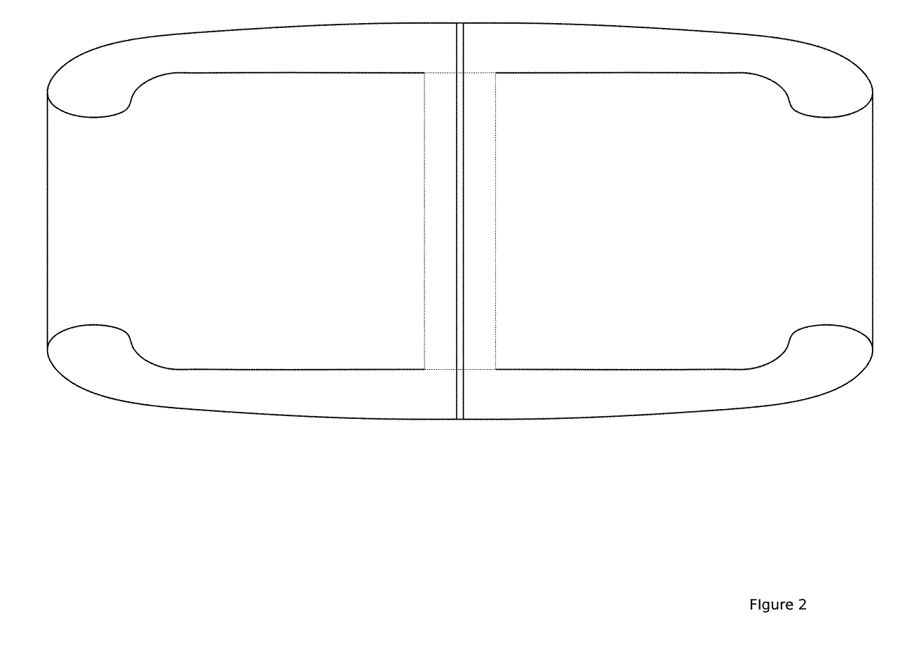

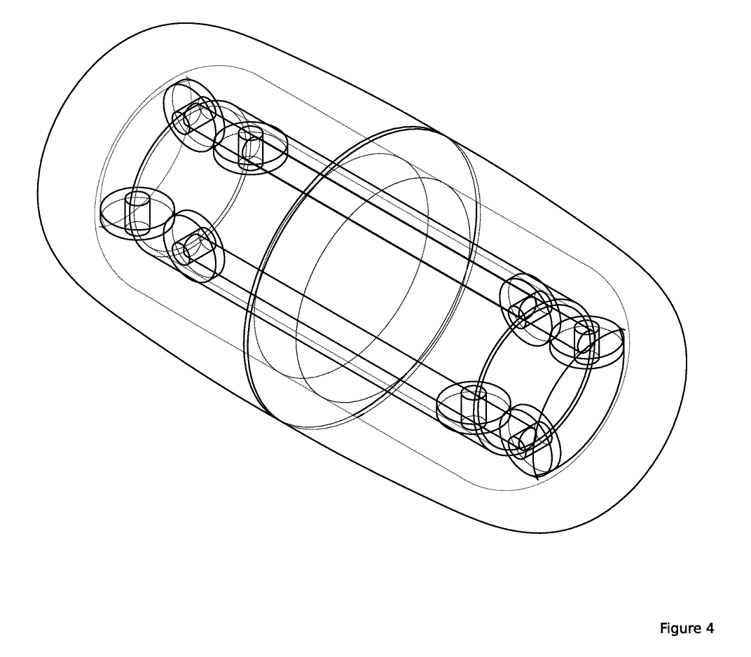

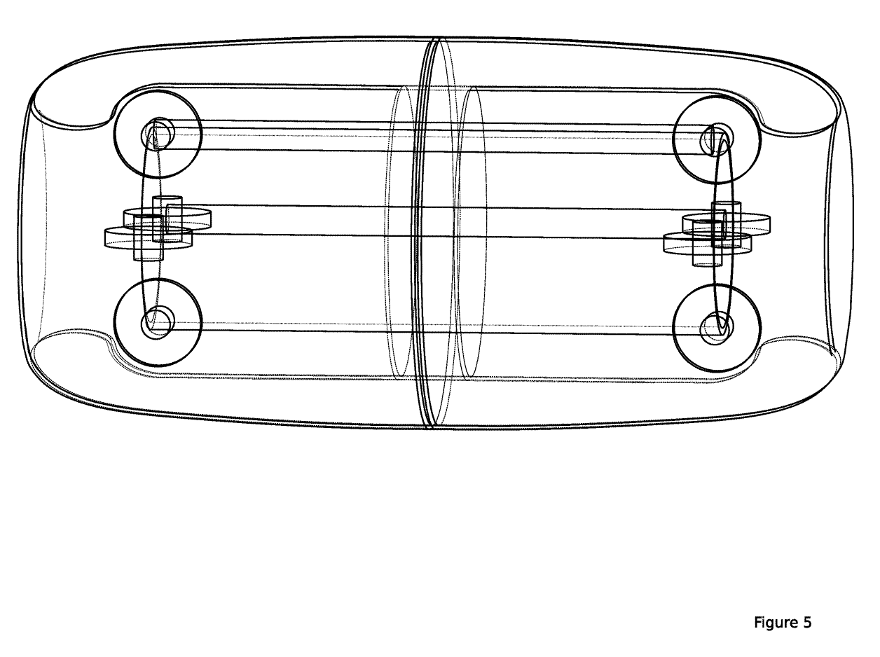

The fabric tube propulsion drive consists of the following components:
1) The endoskeleton: For the Fabric Tube Propulsion Drive, the following physical characteristics must be provided from any specific design implementation of the internal frame of the device, otherwise referred to here on forth as the endoskeleton.
   a) The endoskeleton must provide a low friction surface over which an elastic fabric or mesh can smoothly pass over both the outside and inner surfaces of the tube. The device takes the overall form of a tube once the endoskeleton is ensheathed in a fabric or mesh.
   b) The endoskeleton is flexible such that it can bend in the x or y planes that are perpendicular to the longitudinal axis of the tube.
   c) The endoskeleton is able to contain within the lumen of the tube, a motor drive system that is held in place by narrowings at either end of the tube.
The endoskeleton can take various forms. It can be a one piece mould of variable rigidity polymer such that the tube is rigid over the areas where the motor drive wheels contact the fabric at the inner surface of the tube. Yet flexibility is kept through the middle section(s) of the tube and any intervening areas between the motor or runner wheels. Having circumferential flexiblility in the endoskeleton of the tube allows for lateral flexion of the tube.
Another simple implementation of an endoskeleton is that of an elongated coil spring bounded by low friction end caps. The low friction end caps are moulded to serve the dual purposes of i) providing a smooth surface for the fabric or mesh to traverse over the ends of the tube; and ii) a narrowing to the inner diameter of the tube at either end, which physically constrains a motor system within the inner cavity of the tube. The coil spring endoskeleton provides a general tubular frame structure for the Fabric Tube Propulsion Drive while at the same time providing lateral flexibility.
Please note that FIGS. 1-5 illustrate only one of the possible implementations of the endoskeleton. Any particular design can be used so long as it meets the physical criteria of 1) a), b), and c).
2) Continuous loop of fabric or mesh:
   a) A contiguous loop of fabric surrounds both the inner and outer surfaces of the endoskeleton such that the fabric on the inner surface of the endoskeleton passes over the open ends of the tubular endoskeleton to the outer surface of the endoskeleton.
   b) The fabric or mesh can slide freely over the outer surface of the endoskeleton and around the end of the endoskeleton to the inner surface. Simultaneously as it feeds through the inner surface, that same fabric continuously passes around the other end of the device and over back to the outside of the device.
The fabric or mesh can have various different surface textures to provide adhesion and friction properties appropriate to the body part or cavity being explored. One such surface texture to provide the most efficient and damage free propulsion through the most common body surfaces and cavities is detailed below.
   A pile carpet like surface texturing of the fabric or mesh is ideal for propulsion over and through most body cavities. As the fabric passes along the outside of the device, the fabric or mesh is stretched, allowing for greater separation between the individual tufts of material, hitherto referred to as “piles”. Each “pile” is composed of numerous thin filaments of fibre that provide a soft contact area between the fabric and body surface. Such a soft textured surface allows for a trauma-free interface between the device and body part. The length and stiffness of the individual fibers within each pile can be optimized for the surface characteristics of the body cavity being explored.
   A carpet pile type of textured surface can furthermore allow for propulsion of the device through liquids, as the surface area of the pile covered fabric passing along the outer surface of the tube is greater than the surface area of the fabric passing through the inner surface of the tube. This permits a differential of propulsive forces between the outside of the device and inside of the device allowing for an overall forward propulsive force to be created along the device's longitudinal axis.
3) Motor drive system:
   FIGS. 1-5 illustrate the shortest impelementation of a motor drive system that can provide two axes of lateral flexion of the endoskeleton. Longer implementations of the Fabric Tube Propulsion Drive can have intervening segments of motors and runner wheels added to the middle of the device bewtween the end motors to create whatever length of device necessary for a task. In this manner, the length of the device can be altered to facilitate the device's overall operation, i.e. short implementations may be sufficient for videographic examination of a body part; while longer implementations are necessary for versions of the device that carry surgical tools and mechanisms.

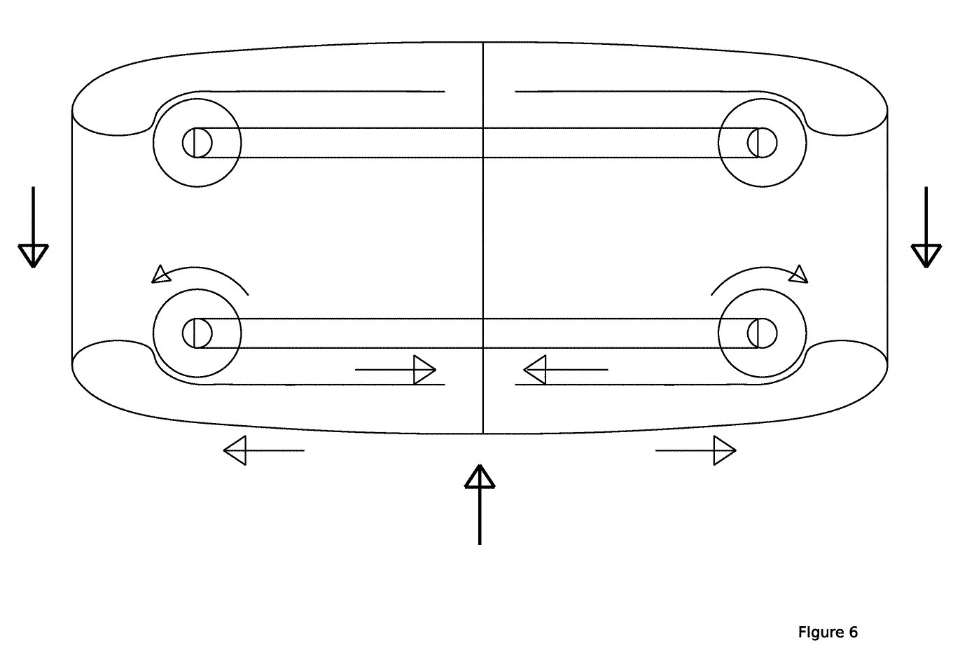

a) Mechanism for lateral flexion of the Fabric Tube Propulsion Drive:
FIGS. 1-6 describe an 8 motor system, with a motor driving each wheel independently. The motors may be mounted on the wheel axles themselves, or perpendicular to the axles with a driveshaft and gearing to rotate each axle.
To create lateral flexion of the endoskeleton, two motors located in the same longitudinal axial plane, but at opposite ends of the device can be used to create tension in the fabric overlying the endoskeleton on the outside of the device by rotating in opposite directions as in FIG. 6. The fabric overlying the outside of the endoskeleton, opposite to the location of the two wheels' contact points on the fabric lining the inner surface of the endoskeleton, is stretched, while the fabric on the inside of the endoskeleton is compressed as indicated by the straight arrows. Deflection of the endoskeleton is indcated by the heavy arrows, i.e. the middle of the device is deflected upwards while the ends of the device are deflected downwards when the wheels are rotated as indicated by the curved arrows. Bending of the endoskeleton can thereby be achieved to varying degrees from localized fabric tension along specific longitudinal planes causing localized compression of the endoskeleton.
Once a certain set level of tension has been achieved by the wheels rotating in opposite directions, the wheels can then rotate in unison in the same rotational direction for propulsive operation of the Fabric Tube Propulsion Drive while the endoskeleton remains in the bent state. This allows the Fabric Tube Propulsion Drive to navigate around corners or point devices such as video cameras or surical tools in specific directions.
For flexion of the Fabric Tube Propulsion Drive device along a perpendicular axis, a perpendicular set of motors is used in the manner described above. However, when size constraints are an issue, such as implementation of the Fabric Tube Propulsion Drive within narrow body cavities, fewer motors and drive wheels can be used. For example, a 6 motor system can be implemented analogously to the 8 motor drive system illustrated in FIGS. 1-6, but with the motors arranged in a triangle (as opposed to a cross) when the device is viewed along its longitudinal axis.

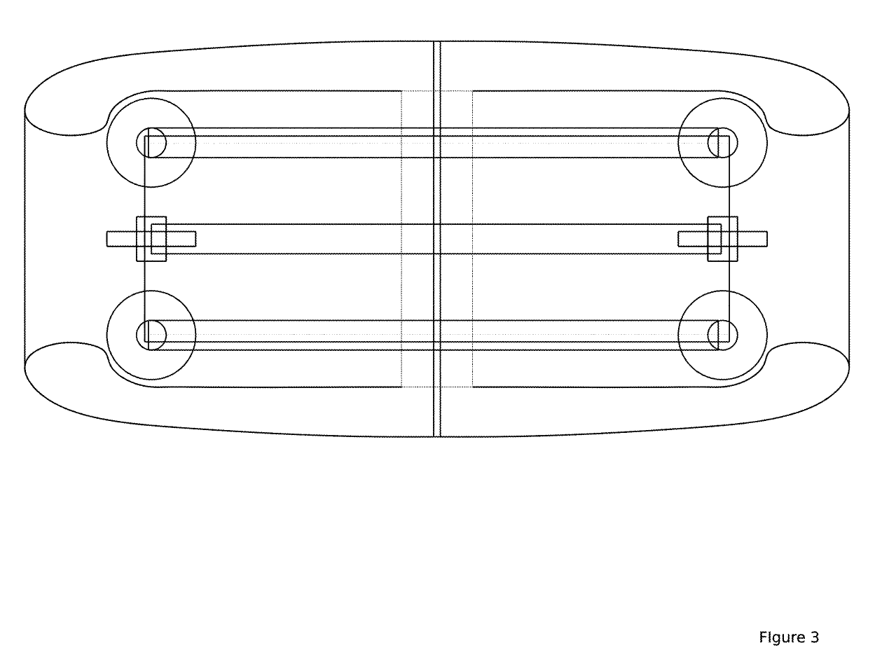

Longer variations of the Fabric Tube Propulsion Drive using an endoskeleton with an elongated midsection may add intervening segments of motor drive and/or passive runner wheels between the drive wheels illustrated in FIGS. 3-6. These intervening segments of passive runner wheels and/or motor drive units sit on a flexible subframe within the lumen of the fabric tube.

b) Mechanism for retention of the motor drive system within the lumen of the endoskeleton:

The motor drive system is mechanically held within the inner lumen of the device by circumferential narrowings of the endoskeleton at both ends of the device. The motor drive system may be permanently manufactured into the device or detachable from the device through one of two methods.
i) a mechanism for expanding the narrowings in the endoskeleton at either end of the device. One such suggested mechanism is a push button clasp that circumferentially unlocks the narrowed end of the endoskeleton, so that it can be widened to free the motor drive system. This push button clasp is situated on the inner surface of the device to avoid accidental actuation during the operation of the device, and is designed such that it can be actuated through the fabric or covering mesh without removing said fabric or covering mesh from the device.
ii) a retraction mechanism for the wheels of the motor drive system that allow the subframe holding motor drive components to be removed through the narrowed end of the endoskeleton.

c) High friction wheels:
The motor drive system utilizes of a set of high friction wheels that interface with the fabric or mesh lining the inner surface of the tube formed by the endoskeleton. As the wheels have no direct contact the the body, they can have any surface that provides the highest traction possible. Furthermore, as the motor drive system is completely contained within the tube of the device, a significant radial outward pressure can be applied to the motor driving wheels to facilitate traction, without any change in the external pressure exerted by the device on the surrounding tissues.

1) An atraumatic propulsion system for moving devices within a human or animal body consisting of a flexible hollow tube surrounded by a continuous loop of textured fabric or mesh that continuously loops around from the inner lumen of the tube to the outside of the tube.

2) A tube shaped inner frame or endoskeleton (over which the continuous loop of elastic fabric or mesh is wrapped) with proximal and distal circumferential narrowings on its inner surface to mechanically constrain a motor drive system.

3) Endoskeleton frame designs using single and multiple materials to provide tubelike structure with the properties of lateral flexibility and longitudinal rigidity for the device described in claim #1.

4) A metal or composite coil spring as the body for the endoskeleton described in claim #3 where plastic or composite end caps provide the circumferential inner surface narrowings as described in claim #2 to mechanically constrain a motor drive system.

5) A pile carpet like surface texture for the elastic fabric or mesh surrounding the tubelike structure described in claims #1 to 4 with tufts of fibre of variable lengths and stiffnesses.

6) A motor drive system mounted on a flexible subframe that sits within the lumen of the tube described in claims #1 to 4.

7) A wheel system that provides high traction to the pile carpet like surface of textured fabric in claim #5 within the lumen of the tube described in claims #1-4.

8) A mechanism for removal of the motor drive system from the inside of the tube described in claims #1 to 4, either through retraction of the wheels away from the circumferential inner narrowings of the tube described in claim #2 and claim #4; or a clasp like system to release and expand the inner circumferential diameter of the endoskeleton at the circumferential narrowings.

9) A method for flexing the tubelike structure described in claims #1 to 4 by locally varying the tension of the elastic fabric or mesh described in claims #1 to 5 with the motor drive system described in claims #6 and 7.
